(19) United States
(12) Patent Application Publication
(10) Pub. No.: US 2012/0301867 A1
(43) Pub. Date: Nov. 29, 2012

(54) RECOVERING NUCLEATED RED BLOOD CELLS AND METHOD FOR CONCENTRATING AND RECOVERING NUCLEATED RED BLOOD CELLS

(75) Inventors: Takeshi Kumo, Nomi-shi (JP); Yuzuru Takamura, Nomi-shi (JP); Haruo Takabayashi, Kahoku-gun (JP)

(73) Assignees: KANAZAWA MEDICAL UNIVERSITY, Ishikawa (JP); Japan Advanced Institute of Science and Technology, Nomi-shi (JP)

(21) Appl. No.: 13/393,854

(22) PCT Filed: Sep. 2, 2010
(86) PCT No.: PCT/JP10/65058

§ 371 Date: Aug. 15, 2012

(30) Foreign Application Priority Data
Sep. 4, 2009 (JP) 2009-205343

(51) Int. Cl. C12M 1/26; C12N 5/078

(52) U.S. Cl. 435/2; 435/309.1

(57) ABSTRACT

Provided is a chip or the like having a particulate concentrating mechanism such as a mechanism that can selectively concentrate nucleated red blood cells contained in maternal blood and derived from a fetus and can collect the concentrated liquid rich in nucleated red blood cells, and also provided is a nucleated red blood cell concentrating/collecting method. A micro-channel chip for concentrating nucleated red blood cells has an inlet-side channel, an outlet-side channel, and a separation narrow channel provided between the inlet-side channel and the outlet-side channel. The separation narrow channel has an inner wall having a dimension through which non-nucleated red blood cells easily pass and nucleated red blood cells hardly pass, and has a means for deforming or moving part of the inner wall of the channel to have a dimension through which nucleated red blood cells easily pass. The following are also provided: a method for collecting a liquid in which nucleated red blood cells obtained by using this chip are concentrated; and a micro-channel chip for concentrating particulates other than for concentrating nucleated red blood cells.

### CROSS-REFERENCE TO RELATED APPLICATIONS

This application claims benefit of priority to Japanese Patent Application No. 2009-205343 filed on Sep. 4, 2009, which is expressly incorporated herein by reference in its entirety.

### TECHNICAL FIELD

The present invention relates to a chip with a micro channel for concentrating a granular material, to a chip for concentrating and recovering nucleated red blood cells, and to a method for concentrating and recovering nucleated red blood cells.

### BACKGROUND ART

Conventional prenatal genetic testing methods not only place physical and mental burdens on the mother, but also entail unavoidable risks of injury and miscarriage of the fetus. Fetal cells (nucleated red blood cells derived from the fetus) are known to travel in blood circulating through the mothers body. If the fetal nucleated red blood cells that are contained in maternal blood could be selectively recovered and the fetal genes analyzed, it would be possible to conduct safe prenatal diagnosis free of risk of injury and miscarriage to the fetus. Using such a method, it would be possible to diagnose fetal genes in the early period of pregnancy, and prepare early on for treatment. Worldwide, about five million prenatal genetic diagnoses are performed each year. Were it possible to practically develop such a safe genetic testing method, it could be anticipated to occupy a large portion of the world market.

However, fetal nucleated red blood cells, which are said to be present in a quantity of about one cell per mL of maternal blood, are not readily recovered. Methods of recovery, such as the use of an antibody recognizing the unique structure of the surface of nucleated red blood cells (an antigen-antibody reaction) and the use of fluorescence-activated cell sorting (FAGS) employing fluorescence-labeled blood cells, are being implemented by the research facilities of various nations, but have all proven inadequate.

One method of recovering nucleated red blood cells that would conceivably afford high reliability is to analyze an image under an optical microscope and recover the nucleated red blood cells detected. However, there is a problem in that a great amount of time is required to detect the single nucleated red blood cell among the several billion cells present in a single mL of blood.

It is anticipated that this problem can be solved by preprocessing a blood sample that has been collected to separate and concentrate the nucleated red blood cells. Currently, inventions and research in which micro-sized structures (pillar structures, pore structures, and the like) are fabricated and used to separate and concentrate the targeted cells (Patent References 1, 2, 3; Non patent References 1, 2, 3) have been reported as methods of separating blood cells using a physical structure as a filter.

### PRIOR ART REFERENCES

### Patent References

[Patent Reference 1] Published Japanese Translation (TOKUHYO) No. 2009-509143 of a PCT International Application (WO2007/035585)
[Patent Reference 2] Published Japanese Translation (TOKUHYO) No, 2008-538283 of a PCT International Application (WO2006/108101)
[Patent Reference 3] Published Japanese Translation (TOKUHYO) No. 2006-501449 of a PCT International Application (WO2004/029221)

### Nonpatent References

[Nonpatent Reference 1] Immunology Letters Vol. 71, pp 5-11, 2000
[Nonpatent Reference 2] Biomolecular Engineering Vol. 21, 00 157-162, 2005
[Nonpatent Reference 3] Journal of Chromatography A, 1162 (2007) 187-192

Patent References 1 to 3 and Nonpatent References 1 to 3 are hereby incorporated in their entirety by reference.

### SUMMARY OF THE INVENTION

### Problem to be Solved by the Invention

In prior art such as that cited above, although the targeted cells are separated or concentrated within the chip, no mechanism for effectively recovering the targeted cells is present within the chip.

For example, in the chip described in Nonpatent Reference 3, cells are separated based on size and cell deformation. There are channels narrowing into four sections 15, 10, 5 and 2.5 μm in width (with a constant depth of 5 μm). When a blood sample containing nucleated red blood cells that are about 8 to 13 μm in diameter is made to flow through the chip, the nucleated red blood cells pass through the channels of width 15 μm, 10 μm, and 5 μm. However, they cannot pass through the 2.5 μm channel, and are held in the first row of 2.5 μm channels. In tests conducted with cord blood, which contains a higher density of nucleated red blood cells than maternal blood, when the first row became clogged with nucleated red blood cells, subsequently arriving nucleated red blood cells passed through an evacuation passage provided between two channels of 2.5 μm, and were recovered by a recovery vessel provided at an exit. This procedure requires 6 to 8 hours. There is no technique for recovering the nucleated red blood cells that have been retained in the first row of 2.5 μm channels, so the recovery efficiency of nucleated red blood cells is extremely poor. Maternal blood contains quite few fetal nucleated red blood cells (on average, about 1.2 cells per mL). When this chip is employed, it is necessary to recover the nucleated red blood cells that have lodged in the first row, but no recovery method is indicated.

Accordingly, one object of the present invention is to provide a chip having a structure that selectively concentrates fetal nucleated red blood cells contained in maternal blood, and permits the recovery of a concentrated liquid rich in nucleated red blood cells; and to provide a chip permitting the recovery of specific granular material from a mixture of granular materials of similar dimensions and differing deformation properties. Another object of the present invention is to provide a method for concentrating and recovering nucleated red blood cells by concentrating nucleated red blood cells from maternal blood and recovering a concentrated liquid rich in nucleated red blood cells.

### Means of Solving the Problem

[1]

A micro channel chip, employed to concentrate a granular material B from a mixture of at least one type of granular material having arbitrary grain diameter and arbitrary deformation property (referred to hereinafter as granular material A) and at least one type of granular material having a larger grain diameter than granular material A and less deformability than granular material A (referred to as granular material B hereinafter),

comprising an inlet side channel, an outlet side channel, and a separation-use narrow channel between the inlet side channel and outlet side channel;

wherein the separation-use narrow channel has an inner wall of dimensions permitting the ready passage of granular material A and tending not to pass granular material B; and

comprising a means of deforming or displacing a part of the inner wall of the channel to achieve dimensions facilitating the passage of granular material B.

[2]

A micro channel chip for concentrating nucleated red blood cells,

comprising an inlet side channel, an outlet side channel, and a separation-use narrow channel between the inlet side channel and outlet side channel;

wherein the separation-use narrow channel has an inner wall of dimensions permitting the ready passage of nonnucleated red blood cells and tending not to pass nucleated red blood cells; and

comprising a means of deforming or displacing a part of the inner wall of the channel to achieve dimensions facilitating the passage of nucleated red blood cells.

[3]

The micro channel chip according to [2], wherein the inner wall of the separation-use narrow channel has a vertical sectional height in the channel in a range of 1 μm to 5 μm, a width in a range of 5 μm to 10 m, and a channel length in a range of 2 μm to 1 m.

[4]

A micro channel chip for concentrating nucleated red blood cells,

comprising an inlet side channel, an outlet side channel, and a separation-use narrow channel between the inlet side channel and outlet side channel;

wherein the separation-use narrow channel has an inner wall of dimensions permitting the ready passage of nonnucleated red blood cells and tending not to pass nucleated red blood cells; and

wherein the dimensions are such that the vertical sectional height in the channel is in a range of 1 μm to 5 μm, the width is in a range of 5 μm to 10 m, and the length of the channel is in a range of 2 μm to 1 m.

[5]

The micro channel chip according to any one of [1] to [3], wherein a plurality of separation-use narrow channels are separated by spacers, the surface of the spacer facing the channel on the outlet side is a curved surface that is convex in shape on the outlet side channel side, and/or the surface of the spacer facing the channel on the inlet side is a curved surface that is convex in shape on the inlet side channel side.

[6]

The micro channel chip according to any one of [1] to [3], [5], wherein the means of deforming or displacing the inner wall of the separation-use narrow channel is comprised of a flexible film disposed as at least part of the inner wall of the separation-use narrow channel, and a pressure-adjustable chamber disposed on the opposite side of the channel from the flexible film.

[7]

The micro channel chip according to [6], wherein the inlet side channel, outlet side channel, and separation-use narrow channel are built into the chip, an inlet connecting to the inlet side channel is present on the chip surface, an outlet connecting to the outlet side channel is present, and an opening connecting to an air chamber is present.

[8]

The micro channel chip according to any one of [1] to [7], wherein the inner wall of each channel is surface treated with a coating to prevent cell adhesion or with a coating to prevent nonspecific adhesion.

[9]

A method for recovering a liquid in which nucleated red blood cells have been concentrated, comprising:

feeding a sample containing nonnucleated red blood cells and nucleated red blood cells from the inlet side channel of the micro channel chip according to any one of [2] to [3], and [5] to [8];

recovering the liquid that has passed through the separation-use narrow channel from the outlet side channel;

causing a part of the inner wall of the channel to deform or displace to assume dimensions allowing ready passage of the nucleated red blood cells,

and while in this state, feeding a recovering liquid from the inlet side channel to recover a liquid rich in nucleated red blood cells from the outlet side channel.

[10]

A method for recovering a liquid in which nucleated red blood cells have been concentrated, comprising

feeding a sample containing nonnucleated red blood cells and nucleated red blood cells to the inlet side channel of the micro channel chip according to [4];

recovering the liquid that has passed through the separation-use narrow channel from the outlet side channel;

feeding a recovering liquid from the inlet side channel or outlet side channel, and

recovering a liquid rich in nucleated red blood cells from the outlet side channel or inlet side channel.

[11]

The method according to [9] or [10], wherein the sample containing the nonnucleated red blood cells and nucleated red blood cells is a fraction that has been recovered with a density of 1.070 to 1.095 g/mL by density gradient centrifugation separation using Percoll.

[12]

The method according to [11], wherein the sample containing the nonnucleated red blood cells and nucleated red blood cells comprises a recovered fraction diluted with a saline solution having a sodium chloride concentration consistent with physiological conditions.

[13]

The method according to any one of [9], [11] to [12], wherein the micro channel chip according to [6] or [7] is employed, and, in the course of feeding the sample containing nonnucleated red blood cells and nucleated red blood cells, the pressure-adjustable chamber is subjected to a positive pressure relative to the separation-use narrow channel to prevent a part of the inner wall of the channel from deforming in a concave manner to the air chamber side.

[14]

The method according to any one of [9], [11] to [13], wherein the micro channel chip according to [6] or [7] is employed and the pressure in the air chamber is reduced relative to the separation-use narrow channel to cause a part of the inner wall of the channel to deform or displace in a concave manner to the air chamber side to achieve dimensions permitting the ready passage of nucleated red blood cells.

[15]

The method according to any one of [9], [11] to [14], wherein the micro channel chip according to [6] or [7] is employed and the pressure in the air chamber is reduced relative to the separation-use narrow channel to cause a part of the inner wall of the channel to deform or displace in a concave manner to the air chamber side to achieve dimensions permitting the ready passage of the liquid through the separation-use channel when the liquid is introduced, during cleaning, or when removing air bubbles.

### Effects of the Invention

The present invention permits the concentration and recovery with high efficiency of the nucleated red blood cells present at extremely low concentration in maternal blood.

### BRIEF DESCRIPTION OF THE DRAWINGS

FIG. 1 Shows an exploded view descriptive of the three-layer structure of one aspect of the chip of the present invention and an enlarged view of channel-forming layer A.

FIG. 2 Shows a sectional view of a surface parallel to the channel descriptive of the three-layer structure of one aspect of the chip of the present invention.

FIG. 3 Schematic descriptive views of working of the intermediate film and the separation of blood cells. The figure on the left shows the air chamber in a slightly positively pressurized state, and the figure on the right shows the air chamber in a negatively pressurized state.

FIG. 4 An image of the gap portion of a channel casting mold made of SU-8 as observed by scanning electron microscopy in Embodiment 1. This photograph is the casting mold; the actual channels are the reverse of the protrusions and indentations.

FIG. 5 Photographs showing density gradient centrifugal separation in Embodiment 2. The left photograph is a test tube containing maternal blood. The center photograph shows a two-fold dilution of the maternal blood with physiological saline. The right photograph shows layered 1.075 g/mL and 1.085 g/mL Percoll solutions.

FIG. 6 Photographs showing density gradient centrifugal separation in Embodiment 2. The left photograph shows the introduction of maternal blood diluted two-fold with physiological saline after layering 1.075 g/mL and 1.085 g/mL Percoll solutions in a test tube. The center photograph was taken after centrifugal separation, with fractionation into various specific gravities. The right photograph shows the recovery of fractions corresponding to specific gravities primarily containing nucleated red blood cells and neutrophils, diluted two-fold with physiological saline.

FIG. 7 Photographs showing density gradient centrifugal separation in Embodiment 2. The left photograph shows the right photograph in FIG. 6 following centrifugal separation. The right photograph was taken after removing the layer containing a large amount of Percoll.

FIG. 8 Photographs showing samples obtained by density gradient centrifugal separation in Embodiment 2. The left photograph shows an image of nucleated red blood cells observed by microscope after staining whole blood by the May-Grunwald-Giemsa staining protocol. The right photograph shows an image of nucleated red blood cells observed by microscope after staining blood cells by the May-Grunwald-Giemsa staining protocol after Percoll centrifugal separation.

FIG. 9 A descriptive drawing showing working of the intermediate film and the release of nucleated red blood cells retained in gaps in the concentration and recovery of nucleated red blood cells in Embodiment 2.

FIG. 10 The appearance (left) of a PDMS chip employed in the concentration and recovery of nucleated red blood cells in Embodiment 2. The right figure is an image of blood cells retained in gaps as observed by microscope after feeding the blood cell sample solution.

FIG. 11 Images showing the concentration and recovery of nucleated red blood cells in Embodiment 2. The left figure is the image of blood cells that have passed through the gaps as observed by microscope after feeding the blood cell sample solution. The right figure is an image as observed by microscope of how blood cells that have remained in the gap are released by causing the intermediate film to work.

FIG. 12 Images of nucleated red blood cells concentrated and recovered in Embodiment 2.

### MODES OF CARRYING OUT THE INVENTION

### [The Micro Channel Chip for Concentrating a Granular Material]

The present invention is a micro channel chip, employed to concentrate a granular material B from a mixture of at least one type of granular material having arbitrary grain diameter and arbitrary deformation property (referred to hereinafter as granular material A) and at least one type of granular material having a larger grain diameter than granular material A and less deformability than granular material A (referred to as granular material B hereinafter). This micro channel chip comprises an inlet side channel, an outlet side channel, and a separation-use narrow channel between the inlet side channel and outlet side channel. The separation-use narrow channel has an inner wall of dimensions permitting the ready passage of granular material A and tending not to pass granular material B. The micro channel chip also comprises a means of deforming or displacing a part of the inner wall of the channel to achieve dimensions facilitating the passage of granular material B.

Using the micro channel chip, it is possible to concentrate and separate granular material B from the mixture of granular materials A and B set forth above. An example of the mixture of granular materials A and B is blood. Specifically, an example of granular material A is nonnucleated red blood cells and an example of granular material B is nucleated red blood cells. Accordingly, one aspect of the micro channel chip employed to concentrate granular materials of the present invention is a micro channel chip for concentrating red blood cells that is used to concentrate nucleated red blood cells from a mixture of nonnucleated red blood cells and nucleated red blood cells.

In the micro channel chip for concentrating granular materials of the present invention, the dimensions of the inner walls of the separation-use narrow channel are such that granular material A passes readily and granular material B tends not to pass. The dimensions of the inner walls of the separation-use narrow channel can be determined as set forth below, for example, so that granular material A passes readily and granular material B tends not to pass.

For example, the dimensions of the inner walls can be denoted as follows. Let h denote the vertical sectional height in the channel. Let w denote the width and let L denote the length of the channel. Denoting the particle diameter of granular material A as φA and the deformation property thereof as dfA, and denoting the particle diameter of granular material B as φB and the deformation property thereof as dfB, the height h, an important element in the performance of the chip, can be set by satisfying the following conditions.

Height h>φA×dfA×k1

Height h<φB×dfB×k1

Here; k1 is a coefficient such that when the case where dfA and dfB remain invariable is denoted as 1, it is possible to set k=1. Particle diameters φA and φB can be suitably selected by known methods, dfA and dfB are primarily determined by the flow rate of the sample liquid in the chip and by the dimensions of the separation-use narrow channel inlet, and can be determined taking into account the stress applied to the granular materials in the separation-use narrow channel inlet. Specifically, dfA and dfB can be obtained by applying an actual sample as a test product on a chip and observing under a microscope the degree to which the granular material deforms in the separation-use narrow channel inlet.

Further, the effect of width w on the ease of passage of granular material A and the difficulty of passage of granular material B is not as great as that of height h. To facilitate the passage of granular material A, it suffices to satisfy at least the following condition:

Width w>φA×(1/dfA)×k2

In this formula, k2 is at least 1. For example, it is desirably 2 to 30. k2 can also be a value greater than 30.

For example, in the micro channel chip for concentrating nucleated red blood cells, granular material A corresponds to nonnucleated red blood cells and granular material B corresponds to nucleated red blood cells. The particle diameter of nonnucleated red blood cells (granular material A) is 4 to 6 μm. However, they are oblate particles about 2 μm in thickness. A thickness of about 2 μm is adopted as φA, and deformation property dfA is 0.4 to 0.6. Nucleated red blood cells (granular material B) vary in their particle diameter by stage. Those handled by the present invention have a particle diameter B of about 8 to 13 μm and a deformation property dfB of about 0.7 to 0.9.

The present invention will be described below by means of the example of a micro channel chip for concentrating nucleated red blood cells.

### [The Micro Channel Chip for Concentrating Nucleated Red Blood Cells]

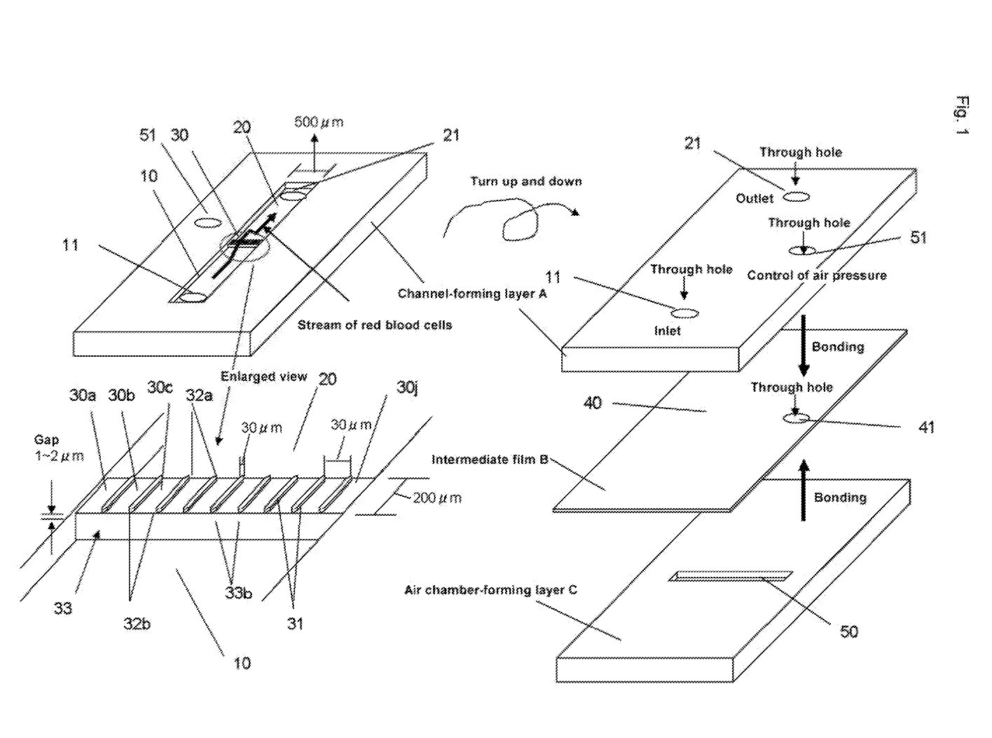

According to FIG. 1, the micro channel chip for concentrating nucleated red blood cells of the present invention is described. The right side of FIG. 1 is an exploded view descriptive of a chip 1 having a three-layer structure comprised of a channel-forming layer A, an intermediate film B, and an air chamber-forming layer C. Inverted upper and lower surfaces views of channel-forming layer A are shown on the upper left. An enlarged view in the vicinity of separation-use narrow channel 30 of channel-forming layer A is shown on the lower left.

Chip 1 comprises an inlet side channel 10, an outlet side channel 20, and a separation-use narrow channel 30 between the inlet side channel and the outlet side channel in channel-forming layer A. Separation-use narrow channel 30 is a narrow channel with dimensions that facilitate the passage of nonnucleated red blood cells and hinder the passage of nucleated red blood cells. The diameter of nonnucleated red blood cells is about 4 to 6 μm, while the diameter of nucleated red blood cells is about 8 to 13 μm. Further, as is also described in Nonpatent Reference 3, cells such as erythrocytes are capable of deforming, and can thus pass through passages that are narrower than the size given above. Specifically, separation-use narrow channel 30 can have a vertical sectional height in the narrow channel that falls within a range of 1 μm to 6 μm, a width falling within a range of 5 μm to 10 m, and a length falling within a range of 2 μm to 1 m. The effect on the separation of nucleated red blood cells of the height in particular among the cross-sectional dimensions is great in the example shown in FIG. 1. Based on the results of the experiments given in the embodiments, the closer the height of the narrow channel is to 1 μm, the greater the recovery rate of nucleated red blood cells becomes, and the closer it approaches 5 μm, the greater the drop in the recovery rate of nucleated red blood cells. The vertical sectional height in the narrow channel desirably falls within a range of 1 to 2 μm. The width can fall within a range of 10 μm to 10 cm, and the length of the channel can fall within a range of 20 to 300 μm. In the micro channel chip for concentrating nucleated red blood cells set forth above, it is possible to cause not just nonnucleated red blood cells, but also white blood cells, to pass through the separation-use narrow channel, and to separate nucleated red blood cells from white blood cells.

As shown in FIG. 1, separation-use narrow channel 30 desirably comprises a plurality of narrow channels 30a, 30b, 30c . . . 30j. For example, separation-use channel 30 can be comprised of 5 to 20 narrow channels. However, the number of narrow channels is not limited. By way of example, the number of narrow channels can range from 1 to 20,000.

Plurality of separation-use narrow channels 30 are separated by spacers 31. The surface 32a of spacer 31 that faces the outlet side of channel 20 is a curved surface of convex shape on the outlet channel side. The surface 32b of the spacer that faces the inlet side of channel 10 is a curved surface of convex shape on the inlet side of the channel. This is desirable so that blood clots tend not to form on the inlet side or outlet side of the spacer, and so as not to impede the passage of the blood sample. Specifically, in terms of design, the curved surfaces of convex shape are semicircles with diameters of 20 to 40 μm. Further, the lateral surface 33b (the surface facing the inlet side of channel 10) of the bank 33 for forming separation-use narrow channel 30 and the lateral surface 33a (the surface facing the outlet side of channel 20, not shown) can also be made similarly convex in shape (with lateral surfaces 33b and 33a being of an overall undulating shape).

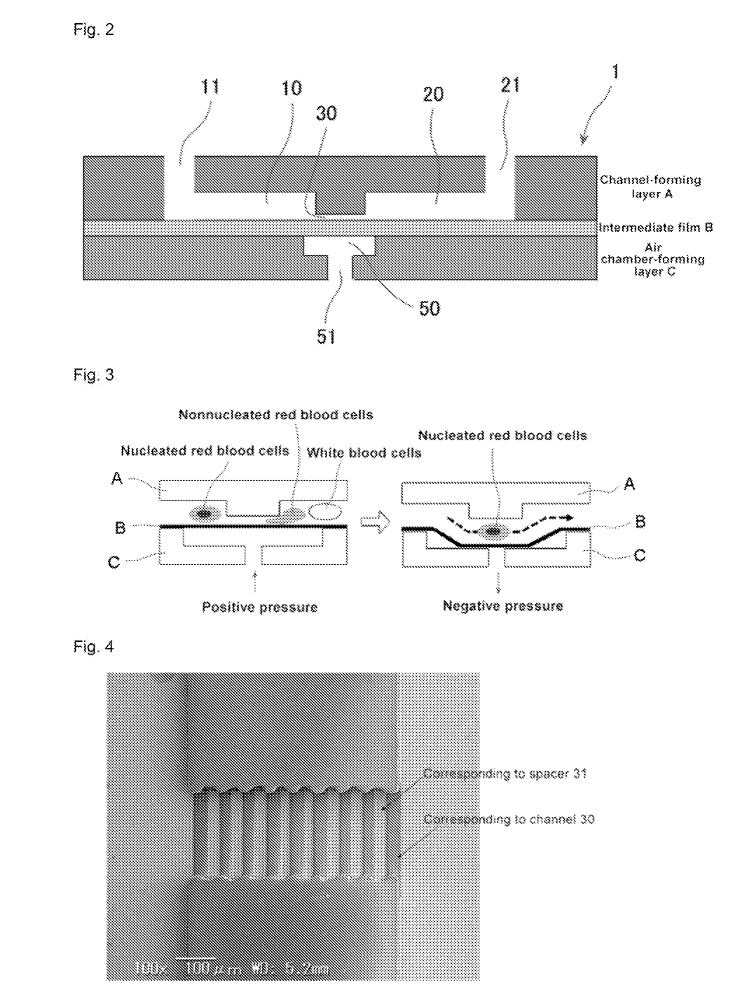

The inlet side of channel 10, the outlet side of channel 20, and separation-use narrow channel 30 are build into chip 1. Inlet 11 connecting to the inlet side of the channel, outlet 21 connecting to the outlet side of the channel, and opening 51 connecting to the air chamber are present on the outer surface of chip 1. As shown in FIGS. 1 and 2 (sectional view), chip 1 can have a three-layer structure comprised of a channel-forming layer A, an intermediate film B, and an air chamber-forming layer C. On one surface, channel-forming layer A comprises inlet side channel 10, outlet side channel 20, and separation-use narrow channel 30 between the inlet side channel and outlet side channel. On the other surface (opposing surface), channel-forming layer A comprises an inlet 11 connecting to the inlet side channel and an outlet 21 connecting to the outlet side channel. On the other surface thereof, channel-forming layer A comprises an opening 51 connecting to the air chamber.

Intermediate film B can have the dimensions of a plane identical to the planar dimensions of channel-forming layer A and air chamber-forming layer C. An opening 41 can be present that connects the space between opening 51 connecting to air chamber 50 present in channel-forming layer A, and air chamber 50 present in air chamber-forming layer C.

Chip 1 also comprises a means of facilitating the passage of nucleated red blood cells through separation-use narrow channel 30 by deforming or displacing a part of the inner wall of narrow channels 30a, 30b, 30c . . . 30j of separation-use narrow channel 30. The means of deforming or displacing the inner wall of the separation-use narrow channel can be comprised of a flexible film 40 provided as at least a part of the inner wall of the separation-use narrow channel and air chamber 50 provided on the opposite side of the flexible film from the channel. Flexible film 40, constituting a portion of intermediate film B, functions as a diaphragm. Positive pressure is exerted on air chamber 50 relative to the channel side, causing flexible film 40 to press against spacer 31 forming separation-use narrow channel 30 and controlling separation-use narrow channel 30 to within the prescribed dimensions set forth above. When the elasticity of intermediate film B itself and the adhesion between the intermediate film and the spacer can maintain the prescribed dimensions, it is possible not to positively pressurize air chamber 50 relative to the channel side. In this state, nucleated red blood cells either cannot pass through separation-use narrow channel 30, or can do so only with difficulty. By contrast, when the pressure in air chamber 50 is reduced, flexible film 40 warps to the air chamber 50 side, the distance between flexible film 40 and the surface of separation-use narrow channel 30 opposite flexible film 40 increases, and it becomes easy for nucleated red blood cells to pass through separation-use narrow channel 30.

The concentration and recovery of nucleated red blood cells using the chip of the present invention will be further described using FIG. 3. Chip 1 of the present invention comprises a narrow channel (micro gap) in which is built a diaphragm drive mechanism comprised of an air chamber 50 and a flexible film 40 functioning as a diaphragm that is deformed by air pressure controls on a portion of separation-use narrow channel 30. A blood sample containing targeted cells (nucleated red blood cells) that is collected from the mother passes through a micro channel having a narrow channel (micro gap), such as that shown on the left in FIG. 3. The nucleated red blood cells (being larger or tending not to deform as much) have greater difficulty passing through the narrow channel than other cells. Thus, the nucleated red blood cells can be selectively trapped at the front of the narrow channel and separated (in case concentration is incomplete, separated) from other cells (mainly nonnucleated red blood cells) that pass more easily along the narrow channel. Subsequently, as shown on the right in FIG. 3, flexible film 40 functioning as a diaphragm is deformed by reducing the pressure in air chamber 50, making it possible to recover the group of cells consisting of concentrated nucleated red blood cells containing the nucleated red blood cells that have been trapped in the front of the narrow channel.

It is suitable for flexible film 40 to be able to keep the narrow channel to within prescribed dimensions in the course of selectively trapping nucleated red blood cells in the front of the narrow channel by increasing the pressure relative of air chamber 50 relative to the channel side, and for flexible film 40 to have physical properties that impart to the narrow channel a gap adequate to allow nucleated red blood cells to pass through the narrow channel when the pressure has been reduced in air chamber 50. From such perspectives, flexible film 40 (or intermediate film B) can be suitably made of silicone resin to impart suitable elasticity and firmness, for example. The phrase “suitable elasticity and firmness” means, for example, resistance to deformation adequate to maintain the dimensions of the gap in the course of generating positive pressure relative to the channel side, and adequate deformation to permit recovery of the nucleated red blood cells in the course of generating negative pressure relative to the channel side. Accordingly, such adequate elasticity and firmness are values that depend on the gap between spacer 31 and spacer 31, and on the size and shape of air chamber 50. The elasticity and firmness of a silicone resin film also varies with the thickness of the film. Thus, it is possible to obtain a film of the desired elasticity and firmness by adjusting the film thickness of the silicone resin in a single material. An example of a silicone resin is polydimethyisiloxane. The gap between spacers 31 is 30 μm, and when air chamber 50 is adequately large, the film thickness can be kept to within a range of 20 to 200 μm, for example.

The inner walls of each channel can be surface treated with a coating agent to prevent cell adhesion or a coating agent to prevent non-specific adhesion, for example. This surface treatment inhibits the adhesion and aggregation of blood cells, platelets, proteins, and the like on the inner walls of the various channels and facilitates the above separation. Examples of coating agents for preventing non-specific adhesion are Blockmaster CE-510 (JSR K.K.), comprising a chief component in the form of polyethylene glycol (PEG), and Lipidure (NOF Corp.).

The present invention covers micro channel chips for concentrating nucleated red blood cells comprising an inlet side channel, an outlet side channel, and a separation-use narrow channel between the inlet side channel and outlet side channel, in which the inner walls of the separation-use narrow channel are of dimensions that facilitate the passage of nonnucleated red blood cells and hinder the passage of nucleated red blood cells, and in which these dimensions are such that the vertical sectional height in the channel falls within a range of 1 μm to 5 μm, the width falls within a range of 5 μm to 10 m, and the length of the channel falls within a range of 2 μm to 1 m. The inlet side channel, outlet side channel, and separation-use narrow channel in micro channel chips of this aspect are identical to the micro channel chip for concentrating nucleated red blood cells of the aspect set forth above. The fact that the dimensions of the inner walls of the separation-use narrow channel facilitate the passage of nonnucleated red blood cells and hinder the passage of nucleated red blood cells, and the fact that these dimensions are such that the vertical sectional height in the channel falls within a range of 1 μm to 5 μm, the width falls within a range of 5 μm to 10 m, and the length of the channel falls within a range of 2 μm to 1 m are identical for the micro channel chip for concentrating nucleated red blood cells of the aspect set forth above.

However, the chip of this aspect does not have a means based on a method of deforming or displacing a part of the inner wall of the channel to facilitate the passage of nucleated red blood cells. When employing a chip of this aspect, the concentration and recovery of nucleated red blood cells are accomplished by feeding a sample containing nonnucleated red blood cells and nucleated red blood cells through an inlet side channel of the micro channel chip, recovering the liquid that passes through the separation-use narrow channel from the outlet side channel, and then feeding the recovering liquid from the inlet side channel or outlet side channel to recover a liquid rich in nucleated red blood cells from the outlet side channel or inlet side channel. That is, the chip of this aspect permits the recovery of a liquid of concentrated nucleated red blood cells by the above method despite not having a means based on a method of facilitating the passage of nucleated red blood cells by deforming or displacing a part of the inner walls of the channel.

### [The Method of Manufacturing the Micro Channel Chip for Concentrating Granular Materials and the Micro Channel Chip for Concentrating Nucleated Red Blood Cells]

Various techniques can be employed to manufacture the chip of the present invention. The technique that is employed is selected in part based on optimal materials. Typical materials for manufacturing the chip of the present invention are glass, silicon, steel, nickel, polymethylmethacrylate (PMMA), polycarbonate, polystyrene, polyethylene, polyolefin, silicons (for example, polydimethylsiloxane), and combinations thereof. Additional materials are known in the technical field. Methods of manufacturing channels from these materials are known in the technical field. These methods include photolithography (such as 3D lithography and X-ray photolithography); molding methods; embossing methods; silicon micromachining methods; wet and dry chemical etching methods: milling methods; diamond cutting methods; lithography, electroplating, and molding (LIGA) methods; and electroplating. For example, with glass, traditional photolithography silicon manufacturing methods followed by wet (KOH) or dry etching (reactive ion etching employing fluorine or some other reactive gas) can be employed. Techniques such as laser microprocessing methods can be adopted for plastic materials with high light absorption efficiency. Since the process in this technique is a continuous one, it is suited to low throughput manufacturing. For mass-produced plastic chips, the thermoplastic injection molding method and compression molding method are suitable. To manufacture the chip of the present invention, the conventional thermoplastic injection molding that is employed to mass produce compact disks (while retaining functional fidelity at the sub-micron level) can be utilized. For example, the functions of the chip can be replicated on a glass master by conventional photolithography. When the glass master is electrically cast, a sturdy, thermal shock-resistant, thermoconductive, hard mold can be produced. This mold then serves as a master template in injection molding or compression molding to mold these functions into plastic chips. Compression molding or injection molding can be selected as the manufacturing method based on the plastic material to be used to manufacture the chip, as well as based on conditions relating to the optical quality and throughput of the final product. The compression molding method (also referred to as the hot embossing method or relief imprinting method) is excellent for small structures, but is difficult to use and has a long cycle time when replicating structures with high vertical/horizontal ratios. However, it affords the advantage of being suited to high molecular weight polymers. The injection molding method works well even on structures with high vertical/horizontal ratios, and is optimal for low molecular weight polymers.

The chip can be manufactured as a single unit or as multiple pieces that are subsequently assembled. In one aspect, each of the layers of channel-forming layer A, intermediate film B, and air chamber-forming layer C of the chip has a channel, through-hole, or the like, as shown in FIG. 1. The layers of the chip can be bonded together by clamps, adhesives, heat, anodic bonding, or surface bonding (such as wafer bonding). Further, chips having channels on more than one flat surface can be fabricated as single pieces by 3D lithography or some other 3D manufacturing technique.

In one aspect, the chip is manufactured by PMMA.

### [The Method for Preparing a Concentrated Liquid of Nucleated Red Blood Cells]

The present invention includes a method for preparing a concentrated solution of nucleated red blood cells. This method for preparing a concentrated solution of nucleated red blood cells employs the micro channel chip of the present invention set forth above. Specifically, it is a method comprising:

(1) feeding a sample containing nonnucleated red blood cells and nucleated red blood cells from the inlet side channel of the chip and recovering the liquid that has passed through the separation-use narrow channel from the outlet side channel; and then
(2) causing a part of the inner wall of the channel to deform or displace to assume dimensions allowing ready passage of the nucleated red blood cells, and while in this state, feeding the recovering liquid from the inlet side channel to recover a liquid rich in nucleated red blood cells from the outlet side channel.

The sample containing nonnucleated red blood cells and nucleated red blood cells is a blood sample containing the targeted cells (nucleated red blood cells) that has been collected from the mother. The size of the blood sample that is collected each time is normally about 5 to 10 mL. In the present invention, the entire quantity or some portion thereof can be employed to recover a liquid rich in nucleated red blood cells.

From the perspective of efficiently recovering nucleated red blood cells, the sample containing nonnucleated red blood cells and nucleated red blood cells is desirably fractionated to obtain a fraction with a high concentration of nucleated red blood cells prior to concentration processing with the chip. The fraction with a high concentration of nucleated red blood cells can be a fraction that has been recovered with a density of 1.070 g/mL to 1.095 g/mL, or 1.075 g/mL to 1.885 g/mL by, for example, density gradient centrifugal separation using Percoll, for example. For the former range, nucleated red blood cells can be recovered from a broad range, while for the latter range, which is narrower than the former range, the ratio of nonnucleated red blood cells present with nucleated red blood cells decreases, enhancing separation efficiency. However, there is no intention to limit the present invention thereto. In addition, methods such as the Ficoll method and red blood cell agglutination method can also be employed to obtain a fraction with a high concentration of nucleated red blood cells. Cells that are larger than or less prone to deformation than nucleated red blood cells, such as white blood cells, are desirably removed in advance by a suitable method. To that end, the dimensions employed in the present invention can be varied.

From the perspective of inhibiting clogging of the narrow channels of the chip by blood cells and inhibiting adhesion of blood cells on the inner walls of the channels, the sample containing nonnucleated red blood cells and nucleated red blood cells is suitably obtained by diluting a fraction that has been recovered with a saline solution having a sodium chloride concentration consistent with physiological conditions. The sodium chloride concentration consistent with physiological conditions falls within a range of 8 to 10 mg/mL, for example. The degree of dilution with a saline solution can be suitably determined by taking into account the flow rate, channel structure, and the like. It is suitable to dilute to a blood cell concentration falling within a range of 1.1×10⁶ to 2.3×10⁶ cells/μL.

The operation of feeding a sample containing nonnucleated red blood cells and nucleated red blood cells from the inlet side channel of the chip and recovering the liquid that has passed through the separation-use narrow channel from the outlet side channel can be conducted by setting the sample feed rate (flow rate) to, for example, within a range of 1 to 100 μL/minute. The sample can be fed using a micro syringe pump (IC3100/KN3319040 Tech-jam) or the like.

The micro channel chip is suitably the chip of the present invention. Specifically, it is suitable to employ a chip in which the means of causing the inner wall of the separation-use narrow channel to deform or displace is comprised of a flexible film provided on at least a part of the inner wall of the separation-use narrow channel and an air chamber provided on the opposite side of the flexible film from the channel; an inlet side channel, outlet side channel, and separation-use narrow channel are built into the chip; and having on the surface of the chip, an inlet connecting to an inlet side channel, an outlet connecting to an outlet side channel, and an opening connecting to the air chamber. Thus, in the course of feeding a sample containing nonnucleated red blood cells and nucleated red blood cells, if necessary, positive pressure relative to the channel side is applied to the air chamber, preventing part of the inner wall of the channel from deforming or displacing in a concave manner to the air chamber side.

Once recovery of the liquid that has passed through the separation-use narrow channel has been completed, a part of the inner wall of the channel is caused to deform or displace to assume dimensions allowing ready passage of the nucleated red blood cells, and while in this state, the recovering liquid is fed from the inlet side channel to recover a liquid rich in nucleated red blood cells from the outlet side channel. Specifically, in the micro channel chip, the pressure in the air chamber is reduced to cause a part of the inner wall of the channel to deform or displace in a concave manner to the air chamber side and assume dimensions facilitating the passage of the nucleated red blood cells. A liquid rich in nucleated red blood cells can be recovered, for example, by employing a saline solution having a sodium chloride concentration consistent with physiological conditions that is employed in the above dilution.

The liquid rich in nucleated red blood cells that is recovered can be used in fetal diagnosis and the like. More specifically, the blood cells are stained by the May-Grunwald-Giemsa staining protocol. To a preparation used for microscopic observation, solution is added dropwise in 2.5 μL quantities. It is then smeared with a glass slide and dried. Subsequently, it is stained by immersion in a glass vessel filled with the stain solution, washed, and dried. The nucleated red blood cells are selected morphologically by observation under a microscope and recovered. Finally, the DNA of the nucleated red blood cells is extracted and genetically analyzed.

The micro channel chip for concentrating granular material of the present invention is not limited to the concentration of nucleated red blood cells. It can be similarly employed to separate and concentrate granular materials of differing size, firmness, and ability to deform. In particular, it can be used to separate and recover leukocytes and erythrocytes. In that case, the red blood cells correspond to granular material A (at least one type of granular material of arbitrary particle diameter and arbitrary deforming property) and the white blood cells correspond to granular material B (at least one type of granular material having a particle diameter greater than that of granular material A and an ability to deform less than that of granular material A). Multiple chips of the present invention with gaps of different dimensions can be used to recover various fractions by connecting them in series. It is particularly effective to separately provide a micro channel chip for concentrating granular material having a gap that does not pass cells that are larger than nucleated red blood cells, particularly leukocytes, upstream from the chip of the present invention. However, as set forth above, the micro channel chip of the present invention can be used to cause the separation-use narrow channel to pass not just nonnucleated red blood cells, but also white blood cells (at which time, nucleated red blood cells do not pass through the separation-use narrow channel). As a result, it is also possible to separate nucleated red blood cells from white blood cells (see Embodiment 3).

In the course of concentration and recovery using the chip of the present invention, it is sometimes necessary to initially introduce a liquid because the micro channels will normally be filled only with air. In this process, air bubbles often form in narrow portions, In that case, they remain in the vicinity of separation-use narrow channel 30, the liquid takes a long time to surmount the narrow portions, and powerful pressure is sometimes needed. In the present invention, at such times, it is possible to deform the diaphragm and readily introduce the liquid.

As set forth above, the present invention includes chips without a means based on a method of deforming or displacing a part of the inner wall of the channel to facilitate the passage of nucleated red blood cells. When employing a chip of that form, nucleated red blood cells can be concentrated and recovered by feeding a sample containing nonnucleated and nucleated red blood cells from the inlet side channel of the micro channel chip, recovering the liquid that has passed through the separation-use narrow channel from the outlet side channel, feeding the recovering liquid from the inlet side channel or outlet side channel, and recovering a liquid rich in nucleated red blood cells from the outlet side channel or inlet side channel. The recovering liquid and the sample containing nonnucleated red blood cells and nucleated red blood cells are identical to those set forth above.

### EMBODIMENTS

The present invention is described in greater detail below through embodiments. However, there is no intent to limit the present invention to the embodiments.

### EMBODIMENT 1

### Chip Preparation

The series of steps up to completion of a chip of polydimethylsiloxane (PDMS) can be roughly divided into designing the channel pattern, preparation of a mask for photolithography, preparation of a channel casting mold, preparation of a PDMS channel layer employing the casting mold, and bonding the various layers.

### Designing the Channel Pattern

The channel pattern was constructed with Illustrator (Adobe) on a PC.

### Preparation of a Mask for Photolithography

The channel pattern was printed on a transparent OHP film. This film was adhered to a transparent glass sheet and a mask for photolithography was completed.

### Preparation of a Channel Casting Mold

Photocuring resist SU-8 (US Micro Chem Corp.) was employed as the material of the channel casting mold. A six-inch silicon wafer with a single surface polished to a mirror finish (CZ-N, Shin-Etsu Chemical Co., Ltd., 625 μm in thickness, crystal plane <100>) was employed as the substrate. The surface of the substrate was cleaned with ultrapure water, dried with nitrogen gas, washed with acetone (EL grade, Kanto Chemical Co., Inc.), and dried again with nitrogen gas. Following cleaning, the substrate was secured to a spin coater (1H-DX2, Mikasa K.K.), a suitable quantity of SU-8 was applied dropwise to the mirror-finished surface side, and spin coating was conducted. Next, the substrate that had been coated with SU-8 was placed on a hot plate (DATAPLATE, AS ONE Corp.), heated for 1 minute at 65° C., and then heated for 1 minute at 95° C. The power to the hot plate was then cut, and the substrate was left standing until the substrate temperature had dropped to about room temperature (pre-baking). A glass mask having the above-described channel pattern was set on a contact exposure-type mask aligner (Suss MJB3 UV400, made by Karl Suss Corp. of the US). UV radiation with a wavelength of 365 nm (i line) was then irradiated onto the SU-8 coated side of the substrate. Following UV exposure, the substrate was placed on the hot plate, heated for 1 minute at 65° C., and heated for 1 minute at 95° C., at which time the power to the hot plate was cut. The substrate was left standing until the substrate temperature had dropped to about room temperature (post-baking). Next, the substrate was immersed for 1 minute 30 seconds in developer (SU-8 Developer, MICRO CHEM) to remove portions of the SU-8 that had not been exposed to UV through the mask. The substrate was then dried with nitrogen gas to complete a channel casting mold of SU-8.

### Preparation of a PDMS Channel Layer Employing the Casting Mold

A SYLGARD 184 silicone elastomer kit (Dow Corning Toray Corp.) was employed with polydimethylsiloxane (PDMS), a silicone resin, as the material. The PDMS main material and a crosslinking agent were mixed in a weight ratio of 10:1. To remove the air that had been entrained during mixing, the PDMS was placed in a bell jar and a vacuum was drawn with a rotary vacuum pump until the bubbles had been completely removed. The debubbled PDMS was caused to flow into an SU-8 channel casting mold on which had been mounted a frame made of silicone rubber to prevent the PDMS from flowing out into the surroundings. Next, it was placed in an oven (DKN 301, Yamato Scientific Co., Ltd.) and left standing for 5 hours at 65° C. to cure the PDMS. The cured PDMS channel was carefully separated from the channel casting mold. A belt hole puncher that had been cleaned with acetone was used to punch holes in necessary spots in the PDMS channel to serve as mounting holes for tubes for controlling the air pressure and for a reservoir for introducing solution.

### Bonding the Various Layers

To remove impurities such as dust that adhered to the PDMS, it was washed with ultrapure water and dried with nitrogen gas. A B layer and a C layer, the surface of which had been cleaned, were oxygen plasma treated for 10 s at 8.8 Pa, 100 W, and 100 sccm with a reactive ion etching apparatus (Reactive Ion Etching-10NR, SAMCO Inc.). While being careful not to touch the surface that had been rendered hydrophilic by the plasma treatment, tweezers were used to handle layers B and C, and a PDMS chip onto which they had been bonded was completed.

As shown in FIGS. 1 and 2, a chip with a three-layer structure comprised of a channel layer, a thin-film layer (intermediate film layer), and an air layer was prepared. In FIG. 1, air inlet 51 of air chamber 50 is provided on the same surface as inlet 11 of channel 10 and outlet 21 of channel 20. By contrast, in FIG. 2, the air inlet 51 of air chamber 50 is provided on the opposite surface from the inlet 11 of channel 10 and the outlet 21 of channel 20. FIG. 2 is a sectional view of the surface parallel with the channel to describe the three-layer structure of the chip. A channel for introducing maternal blood was microprocessed in layer A. Layer B is deformed by applying positive pressure or negative pressure relative to the channel side to the air chamber portion of layer C, and moves vertically in the gap portion in the center of the figure.

A minute gap (microgap) 30 was fashioned in the center of the channel, Holes 5 mm in diameter were fashioned as inlet 11 of channel 10 and outlet 21 of channel 20, and a reservoir was prepared by bonding with intermediate layer B.

FIG. 3 shows schematic descriptive views of working of the intermediate film and the separation of blood cells. It also shows an enlarged view of the vicinity of minute gap (microgap) 30. The figure on the left in FIG. 3 shows the air chamber of layer C in a slightly positively pressurized state relative to the channel, making it possible to prevent the intermediate film of layer B from dropping by means of the pressure generated by flowing of the solution. It shows how blood cells other than nucleated red blood cells that tend to readily deform flow on through the gap. The figure on the right in FIG. 3 shows the state of release for recovering the nucleated red blood cells that have collected by expanding the channel of the gap portion by dropping of the intermediate film of layer B by reducing the pressure of the air chamber of layer C.

As shown in FIG. 3, intermediate film B swings up and down based on the air that is introduced into air chamber 50, and is part of the diaphragm drive. The diaphragm drive is designed to be readily installed by forming air inlet 51 of air chamber 50 on the lower side. The swinging of intermediate film B by driving the diaphragm is achieved by means of the air pressure introduced into air chamber 50.

FIG. 4 is an image of the gap portion of the channel casting mold made of SU-8 as observed by scanning electron microscopy. This photograph is of the casting mold. The actual channel is the reverse of these indentations and protrusions. As shown in FIG. 4, a channel layer branching into a total of 10 channels from a single channel has been fabricated. The channel portions that branch into multiple channels were prepared as gap portions with heights of 1.4 μm or less. The height of the gaps must be such that the nucleated red blood cells that are to be collected in the present embodiment retain. Accordingly, the height of the gaps started at 2.5 μm or less. At a flow rate of 0.1 to 10 μL/min, the nucleated red blood cells retained when the gap height became 1.4 μm. Based on the above, the height of the gaps in the chip was made 1.4 μm or less. The channel spacers were fabricated to inhibit the chip from flexing due to the minute gaps. The spacer inlets in the gap portions were imparted with from square to round (round edged) shapes to inhibit stagnation from occurring when the blood passed by.

### EMBODIMENT 2

Nucleated red blood cells were concentrated and recovered from maternal blood by the following method using the chip fabricated in Embodiment 1.

### <Method of Introducing Blood>

Time was required for the nucleated red blood cells to concentrate on the chip with the quantities of blood collected. Thus, a step to reduce the quantity of blood was necessary. Accordingly, density gradient centrifugal separation was conducted to concentrate nucleated red blood cells and reduce the quantity of blood

### 1. Density Gradient Centrifugal Separation

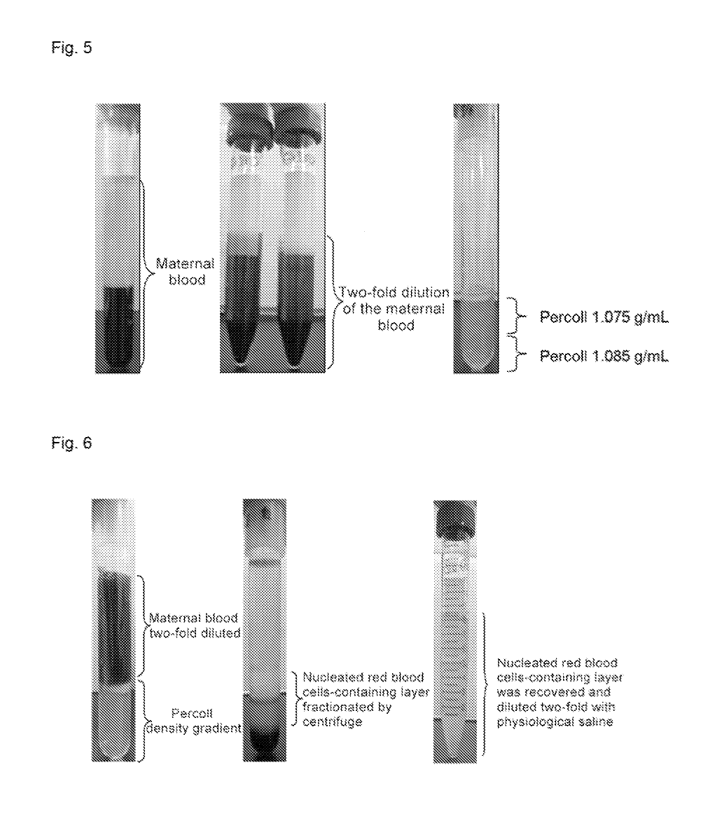

Maternal blood (6.0 mL to 7.0 mL) is collected with a pipette and transferred in two parts to a centrifuge tube. The quantity of maternal blood collected is affected by individual differences in blood viscosity and varied somewhat. However, 6.0 mL or more is consistently collected. The maternal blood that had been divided into two parts is diluted two-fold with a 0.9% (g/mL) NaCl aqueous solution. In a separate centrifuge tube, a density gradient is prepared with Percoll of differing densities (1.075 g/mL, 1.085 g/mL). These results are given in FIG. 5 as a photograph (left) of the test tube to which the material blood was charged, a photograph (middle) taken when the maternal blood was diluted two-fold with physiological saline, and a photograph (right) taken when 1.075 g/mL and 1.085 g/mL Percoll solutions were layered.

The maternal blood that had been diluted two-fold is poured into the centrifuge tube prepared with a density gradient. Centrifugal separation is conducted with a centrifuge (3,000 rpm, 1,750×g, 30 min). The nucleated red blood cell-containing layer that appeared following centrifugation is recovered with a pipette. The nucleated red blood cell-containing layer recovered by pipette is diluted two-fold with a 0.9% (g/mL) NaCl aqueous solution. The results are given in FIG. 6 as a photograph (left) taken when maternal blood is introduced that had been diluted two-fold with physiological saline after layering 1.075 g/mL and 1.085 g/mL Percoll solutions in the test tube; a photograph (middle, with fractionation into various specific gravities) taken following centrifugal separation; and a photograph (right) taken when the fractions corresponding to the specific gravities containing primarily nucleated red blood cells and neutrophils were recovered and diluted two-fold with physiological saline.

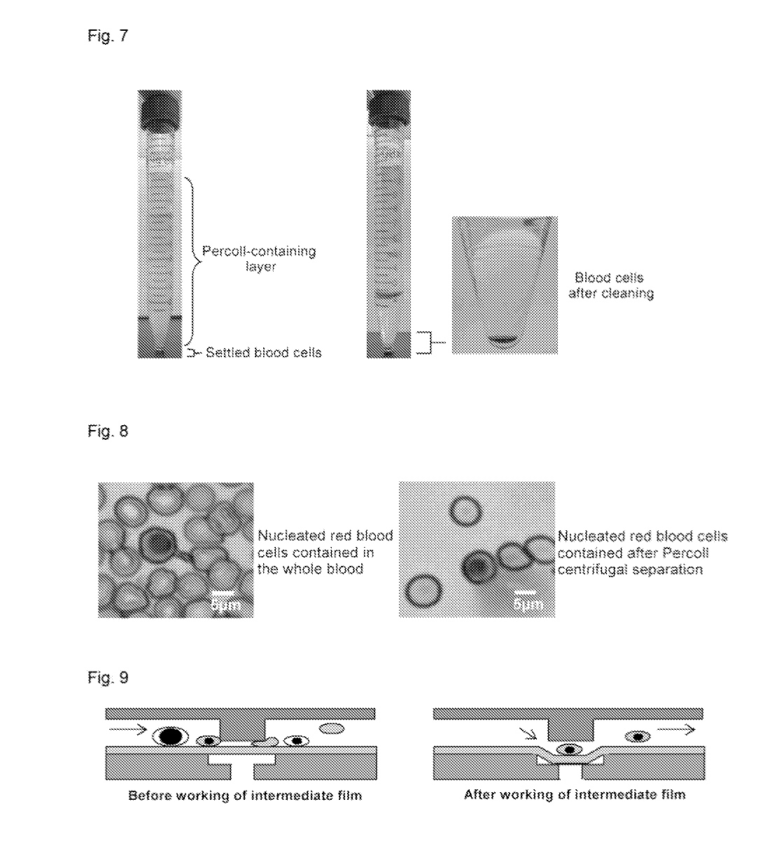

The nucleated red blood cell-containing layer that was recovered after density gradient centrifugal separation and diluted two-fold with 0.9% (g/mL) NaCl aqueous solution contains a large quantity of Percoll. Thus, most of the remaining Percoll is made to move into the top layer of blood cells by conducting centrifugal separation again, and removed with an aspirator. This Percoll removal method is called cleaning. Cleaning is conducted three or more times. The results are given in FIG. 7 as a photograph (left) taken after centrifugal separation of the sample in the photograph on the right in FIG. 6, and a photograph (right) taken after removing the Percoll-containing layer.

The total quantity of blood that had been cleaned and recovered was reduced to about 30 to 60 μL. The entire quantity was reduced to a small quantity. Since the number of blood cells per visual field had decreased, the nucleated red blood cells can be said to have been concentrated. The results are given in FIG. 8. The image on the left was obtained by staining the whole blood by the May-Grunwald-Giemsa staining protocol and observing the nucleated red blood cells under a microscope. The image on the right was obtained by staining the blood cells following Percoll centrifugal separation by the May-Grunwald-Giemsa staining protocol and observing the nucleated red blood cells under a microscope,

### 2. Introducing Blood to the Chip Following Density Gradient Centrifugal Separation

### 2.1 The Principle Behind the Chip

Nucleated red blood cells are recovered with the chip fabricated in Embodiment 1 by causing nucleated red blood cells to retain in the gap portion of the channel in the form of the separation-use narrow channel, causing nonnucleated red blood cells and the like to pass through (left, FIG. 9), and subsequently working the intermediate film (right, FIG. 9). The height of the gap portion was set to 1.0 μm by optimizing the minute gap portion. The blood sometimes settled while passing through the channel. A countermeasure to this was set forth in the optimization of the microgap portion

2.2 Introducing the Blood onto the Chip

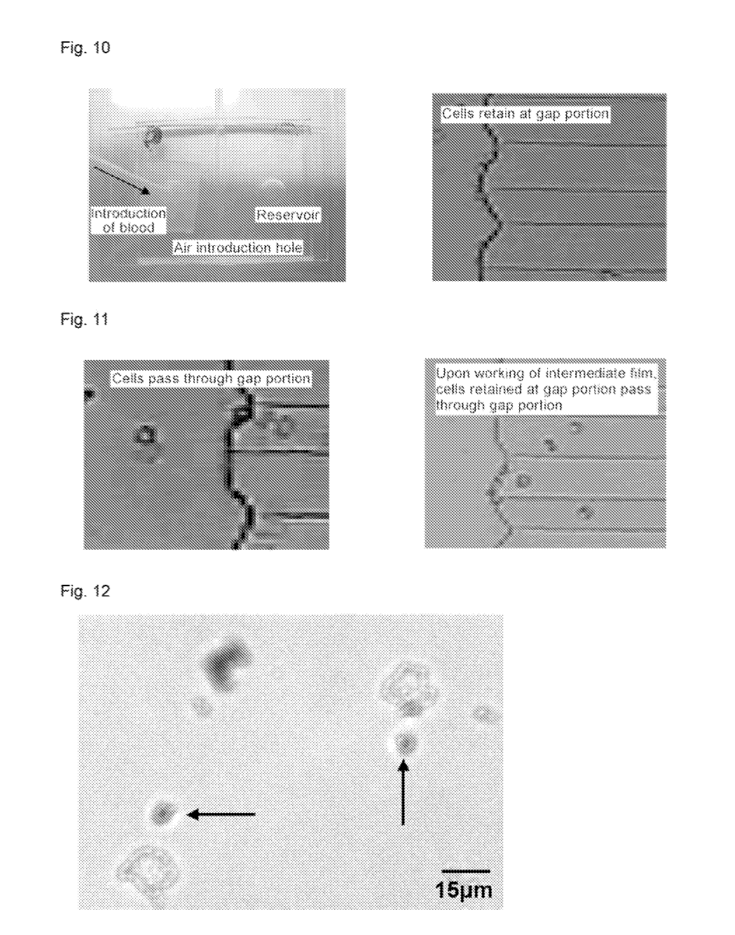

Maternal blood (30 to 60 μL) the quantity of which had been decreased in 2.1 is diluted four-fold and introduced onto the chip. The introduction rate is 10 μL/min. The blood that had passed to the reservoir before working of the intermediate film was sequentially recovered with a micro-pipette. FIG. 10 (left) shows the appearance of the PDMS chip. The right shows an image obtained by feeding the blood cell sample solution and observing the blood cells that retained in the gap portion under a microscope.

To the blood cells that retained in the gap portion is added a 0.9% (g/mL) NaCl aqueous solution and the intermediate film is worked to recover the blood cells in the reservoir. FIG. 11 shows images at that time. The image on the left was obtained by feeding the blood cell sample solution and observing the blood cells that passed through the gap portion under a microscope. The image on the right was obtained by working the intermediate film to release the blood cells that had retained in the gap and observing them under a microscope.

### <The Blood Cells Following Introduction>

The blood cells that had retained in the gap portion inlet were released and recovered from the reservoir. The recovered solution was stained with Giemsa to prepare a specimen. FIG. 12 shows a photograph in which nucleated red blood cells were confirmed by microscope. The arrows indicate nucleated red blood cells.

The above operations were implemented with three chips with gap (separation-use narrow channel) heights of 1.0 μm, 1.4 μm, and 1.85 μm, respectively. The number of nucleated red blood cells detected decreased depending on the gap height. For gap (separation-use narrow channel) heights of 1.0 μm, 1.4 μm, and 1.85 μm, the number of retained nucleated red blood cells was 8 cells, 6 cells, and 3 cells, respectively. The respective recovery rates were 8/8, 6/8, and 3/8. The recovery rate of nucleated red blood cells exhibited its highest value for a gap height of 1.0 μm. That is, the gap height that was advantageous for retaining nucleated red blood cells was 1.0 μm or less. The red blood cells in the samples recovered could not be confirmed. That is thought to be because the red blood cells had passed through the gap portions. For these reasons, it can be said that it was possible to concentrate the nucleated red blood cells because far fewer blood cells other than nucleated red blood cells were present following introduction onto the chip than prior to introduction onto the chip.

### EMBODIMENT 3

The white blood cell and red blood cell elimination rates were determined using the chip of Embodiment 2. First, the original white blood cell count and red blood cell count of 1 mL of maternal blood are determined by FACS. Then, an identical 1 mL of maternal blood is passed over a chip with a microgap of 1.0 μm and the white blood cell count and red blood cell count of the solutions that passed through and are recovered are determined by FACS. The number of blood cells in the solution that passed through the gap is divided by the blood cell count in the original maternal blood and multiplied by 100 to obtain the passage rate (%). The capture rate is calculated as 100−the passing rate. These are determined for the red blood cells and white blood cells. The remaining conditions are identical to those in Embodiment 2.

The results are given below. The original red blood cell count in 1 mL of maternal blood was 3.63×10⁹. The red blood cell count following passage was 3.40×10⁹. On this basis, the red blood cell passage rate was 93.6% and the red blood cell capture rate was 6.34%. The white blood cell count in 1 mL of maternal blood was 1.66×10⁷. The white blood cell count following passage was 1.64×10⁷. On this basis, the white blood cell passage rate was 98.7% and the white blood cell capture rate was 127%.

It merits noting that white blood cells, which are much larger than nucleated red blood cells, are eliminated at a quite high rate. That is attributed to the fact that white blood cells have a live nucleus, are richer in flexibility, and more readily deform than nucleated red blood cells prior to enucleation.

Thus, the removal rate of leukocytes and erythrocytes was about 95% in the present embodiment, making it possible to reduce the overall blood cell count to about one in twenty. That indicates that it is possible to shorten the time required for automated image processing to one-twentieth the current level, which is a tremendous effect.

### INDUSTRIAL APPLICABILITY

The present invention is useful in the fields of the manufacturing and use of chips for concentrating nucleated red blood cells.

### KEY TO THE NUMBERS

1 Chip
10 Inlet side channel
11 Inlet connecting to inlet side channel
20 Outlet side channel
21 Outlet connecting to outlet side channel
30 Separation-use narrow channel
31 Spacer
40 Flexible film (intermediate film B)
50 Air chamber
51 Opening connecting to air chamber

1. A micro channel chip, employed to concentrate a granular material B from a mixture of at least one type of granular material having arbitrary grain diameter and arbitrary deformation property (referred to hereinafter as granular material A) and at least one type of granular material having a larger grain diameter than granular material A and less deformability than granular material A (referred to as granular material B hereinafter),
comprising an inlet side channel, an outlet side channel, and a separation-use narrow channel between the inlet side channel and outlet side channel;
wherein the separation-use narrow channel has an inner wall, at least a part of which is comprised of a flexible film and an air chamber is provided on the opposite side of the flexible film from the air channel and the inner wall has dimensions permitting the ready passage of granular material A and tending not to pass granular material B; and
comprising a means of deforming the flexible film by adjusting the pressure in the air chamber to achieve dimensions facilitating the passage of granular material B.

2. A micro channel chip for concentrating nucleated red blood cells,
comprising an inlet side channel, an outlet side channel, and a separation-use narrow channel between the inlet side channel and outlet side channel;
wherein the separation-use narrow channel has an inner wall, at least a part of which is comprised of a flexible film and an air chamber is provided on the opposite side of the flexible film from the channel and the inner wall has dimensions permitting the ready passage of nonnucleated red blood cells and tending not to pass nucleated red blood cells; and
comprising a means of deforming or displacing a part of the inner wall the flexible film by adjusting the pressure in the air chamber to achieve dimensions facilitating the passage of nucleated red blood cells.

3. The micro channel chip according to claim 2, wherein the inner wall of the separation-use narrow channel has a vertical sectional height in the channel in a range of 1 μm to 2 μm, a width in a range of 10 μm to 10 cm, and a channel length in a range of 20 μm to 300 μm.

4. A micro channel chip for concentrating nucleated red blood cells,
comprising an inlet side channel, an outlet side channel, and a separation-use narrow channel between the inlet side channel and outlet side channel;
wherein the separation-use narrow channel has an inner wall of dimensions permitting the ready passage of nonnucleated red blood cells and tending not to pass nucleated red blood cells; and
wherein the dimensions are such that the vertical sectional height in the channel is in a range of 1 μm to 2 μm, the width is in a range of 10 μm to 10 cm, and the length of the channel is in a range of 20 μm to 300 μm.

5. The micro channel chip according to claim 1, wherein a plurality of separation-use narrow channels are separated by spacers, the surface of the spacer facing the channel on the outlet side is a curved surface that is convex in shape on the outlet side channel side, and/or the surface of the spacer facing the channel on the inlet side is a curved surface that is convex in shape on the inlet side channel side.

6. The micro channel chip according to claim 1, wherein the inlet side channel, outlet side channel, and separation-use narrow channel are built into the chip, an inlet connecting to the inlet side channel is present on the chip surface, an outlet connecting to the outlet side channel is present, and an opening connecting to an air chamber is present.

7. The micro channel chip according to claim 1, wherein the inner wall of each channel is surface treated with a coating to prevent cell adhesion or with a coating to prevent nonspecific adhesion.

8. The micro channel chip according to claim 4, wherein the inner wall of each channel is surface treated with a coating to prevent cell adhesion or with a coating to prevent nonspecific adhesion.

9. A method for recovering a liquid in which nucleated red blood cells have been concentrated, comprising:
feeding a sample containing nonnucleated red blood cells and nucleated red blood cells from the inlet side channel of the micro channel chip according to claim 2;
recovering the liquid that has passed through the separation-use narrow channel from the outlet side channel;
causing a part of the inner wall of the channel to deform or displace to assume dimensions allowing ready passage of the nucleated red blood cells,
and while in this state, feeding a recovering liquid from the inlet side channel to recover a liquid rich in nucleated red blood cells from the outlet side channel.

10. A method for recovering a liquid in which nucleated red blood cells have been concentrated, comprising
feeding a sample containing nonnucleated red blood cells and nucleated red blood cells to the inlet side channel of the micro channel chip according to claim 4;
recovering the liquid that has passed through the separation-use narrow channel from the outlet side channel;
feeding a recovering liquid from the inlet side channel or outlet side channel, and
recovering a liquid rich in nucleated red blood cells from the outlet side channel or inlet side channel.

11. The method according to claim 9, wherein the sample containing the nonnucleated red blood cells and nucleated red blood cells is a fraction that has been recovered with a density of 1.070 g/mL to 1.095 g/mL by density gradient centrifugation separation using Percoll.

12. The method according to claim 11, wherein the sample containing the nonnucleated red blood cells and nucleated red blood cells comprises a recovered fraction diluted with a saline solution having a sodium chloride concentration consistent with physiological conditions.

13. The method according to claim 9, wherein in the course of feeding the sample containing nonnucleated red blood cells and nucleated red blood cells, the pressure-adjustable chamber is subjected to a positive pressure relative to the separation-use narrow channel to prevent a part of the inner wall of the channel from deforming in a concave manner to the air chamber side.

14. The method according to claim 9, wherein the pressure in the air chamber is reduced relative to the separation-use narrow channel to cause a part of the inner wall of the channel to deform in a concave manner to the air chamber side to achieve dimensions permitting the ready passage of nucleated red blood cells.

15. The method according to claim 9, wherein the pressure in the air chamber is reduced relative to the separation-use narrow channel to cause a part of the inner wall of the channel to deform in a concave manner to the air chamber side to achieve dimensions permitting the ready passage of the liquid through the separation-use channel when the liquid is introduced, during cleaning, or when removing air bubbles.

16. The micro channel chip according to claim 2, wherein a plurality of separation-use narrow channels are separated by spacers, the surface of the spacer facing the channel on the outlet side is a curved surface that is convex in shape on the outlet side channel side, and/or the surface of the spacer facing the channel on the inlet side is a curved surface that is convex in shape on the inlet side channel side.

17. The micro channel chip according to claim 2, wherein the inlet side channel, outlet side channel, and separation-use narrow channel are built into the chip, an inlet connecting to the inlet side channel is present on the chip surface, an outlet connecting to the outlet side channel is present, and an opening connecting to an air chamber is present.

18. The micro channel chip according to claim 2, wherein the inner wall of each channel is surface treated with a coating to prevent cell adhesion or with a coating to prevent nonspecific adhesion.

19. The method according to claim 10, wherein the sample containing the nonnucleated red blood cells and nucleated red blood cells is a fraction that has been recovered with a density of 1.070 g/mL to 1.095 g/mL by density gradient centrifugation separation using Percoll.
